(19) United States
(12) Patent Application Publication
(10) Pub. No.: US 2022/0323783 A1
(43) Pub. Date: Oct. 13, 2022

(54) SYSTEMS, APPARATUSES AND METHODS OF UTILIZING BLUE-LIGHT THERAPY FOR TREATMENT AND MITIGATION OF INSOMNIA

(71) Applicant: Click Therapeutics, Inc., New York, NY (US)

(72) Inventor: Anthony Martorell, New York, NY (US)

(73) Assignee: Click Therapeutics, Inc., New York, NY (US)

(21) Appl. No.: 17/639,917

(22) PCT Filed: Sep. 4, 2020
(86) PCT No.: PCT/US2020/049435

§ 371 Date: Mar. 3, 2022

Related U.S. Application Data
(60) Provisional application No. 62/895,931, filed on Sep. 4, 2019.

(51) Int. Cl. A61N 5/06

(52) U.S. Cl. CPC A61N 5/0613; A61N 2005/0663; A61N 2005/0626

(57) ABSTRACT

A computer system for blue-light therapy comprising one or more processors, one or more computer-readable memories, and one or more computer-readable storage devices, and program instructions stored on at least one of the one or more storage devices for execution by at least one of the one or more processors via at least one of the one or more memories, the stored program instructions including determining a need for blue-light therapy, and providing a blue-light stimulus to a user, wherein the blue-light stimulus comprises a visible range of 380 to 500 nm, and wherein, in response to the blue-light stimulus, a circadian rhythm of the user is shifted to a second rhythm, the second rhythm different than the circadian rhythm prior to the administration of the blue-light stimulus.

### PRIORITY

The present application claims priority to U.S. Provisional Patent Application No. 62/895,931, which was filed in the United States Patent and Trademark Office on Sep. 4, 2019, the entire disclosure of which is incorporated herein by reference.

### INTRODUCTION

In mammals, internal biological clocks (e.g. sleep-wake cycles, temperature, blood pressure, immune function, etc.) are regulated by exogenous factors such as environmental-time cues. Normally, such biological clocks are synchronized to a twenty-four hour light-dark cycle. This cycle is based on environmental factors, and referred to as a circadian rhythm.

The circadian rhythm is encoded in biology, and often exhibits even without normal light-dark cycles. Thus, even without exposure to the “outside world,” a person continues to remain bound by their “clock,” awaking at what would be a daytime period and sleeping at what would be a nighttime period.

Positive and negative-feedback elements from circadian “clock” genes are also responsible for regulating various circadian parameters, such as its phase and duration, and are the reason why mutations can have drastic effects on sleep behavior. The circadian rhythm is governed in a timekeeping center of the brain called the suprachiasmatic nucleus (SCN) of the hypothalamus, and is connected to the environment (light input) through the monosynaptic retinohypothalamic pathway.

Influenced by changes in light-dark cycles, the SCN regulates the production of melatonin synthesized from the pineal gland. The retinohypothalamic pathway relays information regarding light to the SCN via ganglionic photoreceptor cells in the retina, which uniquely detect blue light in the range of 460-470 nm. Blue light detection suppresses the production of melatonin. And Melatonin hastens onset of the sleep cycle. Thus, biosynthesis of melatonin predominantly occurs at night when blue light is not as significantly visible.

It is hypothesized that the blue light range of the visible white light spectrum is responsible for the modulating effect of the light-dark cycles on the SCN. Melatonin exerts its physiological actions through melatonin receptor subtypes in both neural and peripheral tissues. The MT₁ receptor decreases neuronal firing rates in the SCN, whereas the MT₂ receptor regulates phase shifts. The actions of melatonin on both MT₁ and MT₂ receptors plays a substantial role in sleep induction and the sleep-wake cycle, including the regulation of core body temperature.

Desynchronization of circadian rhythms can result in disturbances in sleep patterns, including insomnia characteristics such as delayed sleep phase syndrome (DSPS), advanced sleep phase syndrome (ASPS), and shift-work sleep disorder (SWSD). DSPS occurs when sleep onset and wake time are delayed, in some cases by 2-6 hours. DSPS is characterized by a chronic inability to fall asleep at the desired clock time, with patients demonstrating abnormal phase positioning of melatonin. ASPS is characterized by persistent early evening sleep onset and early morning awakening, and has been attributed to age-related attenuation of the rhythm of melatonin secretion. SWSD occurs in rotating-shift workers which forego nocturnal sleep while they are on a night shift, thus having to sleep during the daytime. Chronic inversions of the sleep-wake rhythm confer general insomnia-related symptoms.

Various pharmacological and non-pharmacological treatment methods may be used for difficulty falling asleep at appropriate clock hours. Although some intervention treatments have indicated improvements in sleep (e.g., cognitive behavioral therapy, melatonin supplements, relaxation techniques), a need exists for a therapeutic to directly modulate a disputed circadian rhythm in a timely manner.

It would be desirable, therefore, to provide a therapeutic that modulates a disrupted circadian rhythm in a timely manner.

It would be further desirable to provide a method of utilizing blue light therapy to modulate a disrupted circadian rhythm.

### BRIEF DESCRIPTION OF THE DRAWINGS

FIG. 1 illustrates a block diagram of a distributed computer system that can implement one or more aspects of an embodiment of the present invention;

FIG. 2 illustrates a block diagram of an electronic device that can implement one or more aspects of an embodiment of the invention; and

FIGS. 3A-3P show source code that can implement one or more aspects of an embodiment of the present invention.

While the invention is described with reference to the above drawings, the drawings are intended to be illustrative, and the invention contemplates other embodiments within the spirit of the invention.

### DETAILED DESCRIPTION

The present invention will now be described more fully hereinafter with reference to the accompanying drawings which show, by way of illustration, specific embodiments by which the invention may be practiced. This invention may, however, be embodied in many different forms and should not be construed as limited to the embodiments set forth herein; rather, these embodiments are provided so that this disclosure will be thorough and complete, and will fully convey the scope of the invention to those skilled in the art. Among other things, the present invention may be embodied as devices or methods. Accordingly, the present invention may take the form of an entirely hardware embodiment, an entirely software embodiment, or an embodiment combining software and hardware aspects. The following detailed description is, therefore, not to be taken in a limiting sense.

Throughout the specification and claims, the following terms take the meanings explicitly associated herein, unless the context clearly dictates otherwise. The phrases “in one embodiment,” “in an embodiment,” and the like, as used herein, does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” includes plural references. The meaning of “in” includes “in” and “on.”

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

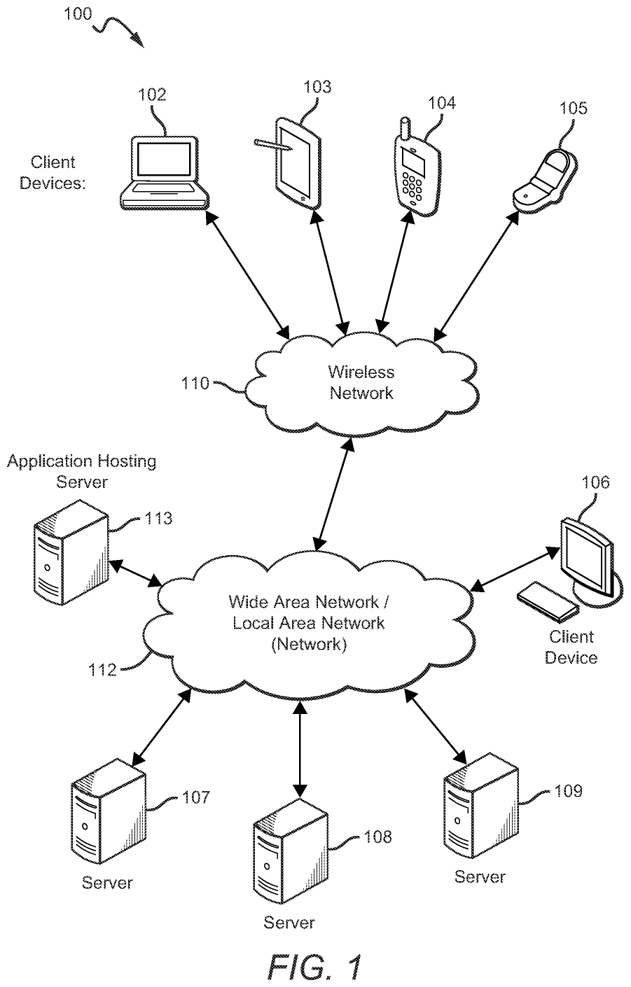

FIG. 1 illustrates components of one embodiment of an environment in which the invention may be practiced. Not all of the components may be required to practice the invention, and variations in the arrangement and type of the components may be made without departing from the spirit or scope of the invention. As shown, the system 100 includes one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. Various of the client devices 102-106 may include, for example, desktop computers, laptop computers, set top boxes, tablets, cell phones, smart phones, smart speakers, wearable devices (such as the Apple Watch) and the like. The servers 107-109 can include, for example, one or more application servers, content servers, search servers, and the like. FIG. 1 also illustrates application hosting server 113.

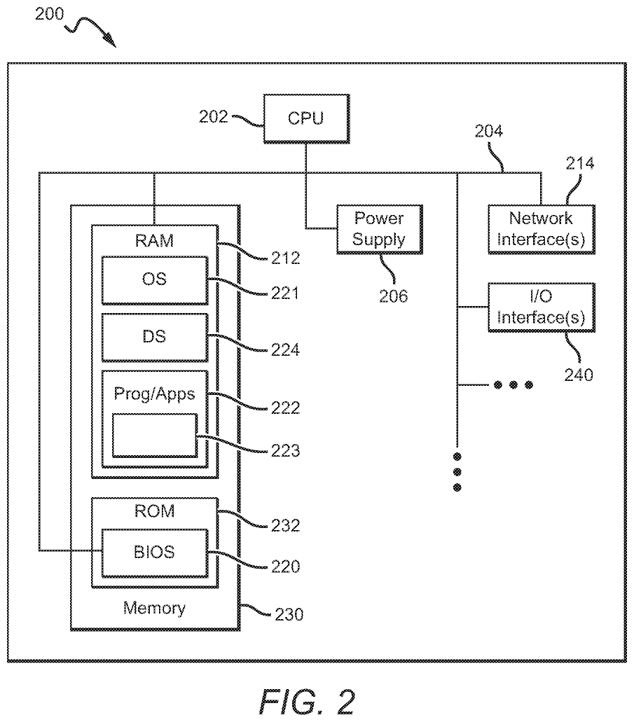

FIG. 2 illustrates a block diagram of an electronic device 200 that can implement one or more aspects of systems, apparatuses and methods for utilizing blue light therapy for treatment and mitigation of insomnia (the “Therapy”) according to one embodiment of the invention. Instances of the electronic device 200 may include servers, e.g., servers 107-109, and client devices, e.g., client devices 102-106. In general, the electronic device 200 can include a processor/CPU 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 200. A touchscreen may determine whether a user is providing input by, for example, determining whether the user is touching the touchscreen with a part of the user's body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the Therapy program 223. The ROM 232 can also include Basic Input/Output System (BIOS) 220 of the electronic device.

Software aspects of the Therapy program 223 are intended to broadly include or represent all programming, applications, algorithms, models, software and other tools necessary to implement or facilitate methods and systems according to embodiments of the invention. The elements may exist on a single computer or be distributed among multiple computers, servers, devices or entities.

The power supply 206 contains one or more power components, and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can ease processing performed by the processor 202.

Where the electronic device 200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Therapy, via a network to another device. Also, an application server may, for example, host a web site that can provide a user interface for administration of example aspects of the apparatus, system and method for the Therapy.

Any computing device capable of sending, receiving, and processing data over a wired and/or a wireless network may act as a server, such as in facilitating aspects of implementations of the apparatus, system and method for the Therapy. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method for the Therapy. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Therapy. Content may include, for example, text, images, audio, video, and the like.

In example aspects of the apparatus, system and method embodying the Therapy, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method embodying the Therapy, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method implementing the Therapy, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games (such as fantasy sports leagues), receiving advertising, watching locally stored or streamed video, or participating in social networks.

In example aspects of the apparatus, system and method implementing the Therapy, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Therapy, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Therapy, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present invention include apparatuses, systems, and methods implementing the Therapy. Embodiments of the present invention may be implemented on one or more of client devices 102-106, which are communicatively coupled to servers including servers 107-109. Moreover, client devices 102-106 may be communicatively (wirelessly or wired) coupled to one another. In particular, software aspects of the above may be implemented in the Therapy program 223. The Therapy program 223 may be implemented on one or more client devices 102-106, one or more servers 107-109, and 113, or a combination of one or more client devices 102-106, and one or more servers 107-109 and 113.

Insomnia is characterized by disrupted sleep patterns, which can manifest as difficulty falling asleep or difficulty remaining asleep. Insomnia may be classified broadly as either cognitive or physiological-based insomnia.

In certain instances of physiological-based insomnia, dysregulation of circadian rhythms may be a cause of sleep difficulty. The dysregulation may result from a variety of reasons, including mutations in clock genes responsible for the regulation of the awake-sleep cycle, as well as a shifted circadian pacemaker, where the insomnia subject naturally falls asleep much earlier or later than normal and thus wakes up at inappropriate times for a typical day-time schedule. Over time, this dysregulation will result in tiredness and “sleep debt” during the daytime. Alternatively, the dysregulation may result in abnormal levels of beta activity during sleep periods. For example, 12-30 Hz beta activity during sleep periods may correlate with periods of hyperarousal, where the insomnia subject cannot fall asleep, and/or is easily awoken.

In an embodiment, apparatuses, systems, and methods implementing the Therapy may be implemented at least in part in software aspects of the Therapy program 223.

Provided herein are systems, apparatuses and methods for utilizing blue light therapy for directly modulating a disrupted circadian rhythm.

In an embodiment, the therapy causes a shifting of the circadian rhythm toward a desired sleep time. Blue light therapy may be administered by exposing a user to a source of blue light. For example, a smartphone, smartwatch, tablet or any other suitable device may be programmed to display blue light. Thus, the device may be programmed to display light with a visible range of 380 to 500 nm (blue light), light with a visible range of 380 to 450 nm (blue-violet light), or light with a visible range of 450 to 500 nm (blue-turquoise light).

The device may then display the programmed light in the visible range, for a specified period of time (for example, 15 or 30 minutes, or any other suitable period). The device may be further programmed to expose the user to the blue light source at a specified time, such as, for example, 10 PM, or right before a user attempts sleep.

In an embodiment, if the user experiences difficulty falling asleep, blue light may be administered immediately upon waking up, in order to shift their circadian rhythm backward. Specifically, by administering blue light after a sleeping event, the circadian rhythm would be shifted backward.

In a further embodiment, if a user has difficulty staying asleep at night and therefore awakes too early in the morning, the therapy may be administered before going to sleep at night, thereby shifting the circadian rhythm forward and reducing the difficulty in staying awake during the day.

In an embodiment, a camera on the device may monitor and assess pupil constriction of an eye of a user. The pupil constriction may be used to assess whether the therapy is being properly received, with the constriction indicating the therapy is being received. Thus, if the camera determines that the pupils are not constricted to a predetermined level, the duration of blue light may be adjusted to a longer duration. Alternatively, if the camera determines that the pupils are properly constricted, duration of blue light may be adjusted to a shorter duration.

In an illustrative process, blue light therapy may include the following sequence: (1) user tracking of number of hours slept and/or monitoring, by a wearable monitor communicatively coupled to a smartphone or other device such as a server, of number of hours slept by user by, for example, monitoring user heart rate (pulse), blood pressure, body temperature, maximum rate of decline body temperature, body movement patterns, mobility, respiration rate, and other suitable indicators; (2) user input of sleep habits and levels, to provide for personalized blue light therapy administration, including whether they are DSPS, ASPS, night-shift worker, and sleep hygiene habits; and (3) blue light stimulation before and/or after a sleep period.

In accordance with an embodiment, suitability of blue light therapy may be determined by analyzing a user's eyes for presence of eye disorders or vision problems, and intermittent sleep-awakening. Eye disorders or vision problems may be assessed by accessing the camera in the device, capturing images of one or both eyes, and comparing the eyes to a library of indicators for a plurality of disorders. The comparison may be conducted by, for example, a trained neural network running on the device or on a server communicatively coupled to the device. In another embodiment, eye disorders or vision problems may be determined by analyzing pupil response, iris or corneal damage, light reflection, or any other suitable indicator. Eye disorders or vision problems may also be determined by the device analyzing a user's medical records. Intermittent sleep-awakening may be determined by analyzing sleep patterns, such as through the device's sleep tracker, and providing a flag to indicate the unsuitability of blue light therapy if intermittent sleep-awakening is found.

In one embodiment, at an initial stage, before commencing nighttime blue-light treatment, a user is exposed to a brief period of blue light on a device during the day, to acquaint their body to the blue light treatment. The user may then determine whether discomfort exists. If there is discomfort, the user may repeat the process on subsequent days, for example, one to three more times. If no discomfort then exists, the user may then continue the treatment. If discomfort persists, the user may not proceed with the treatment.

In an embodiment, a user is exposed to blue light for a specified period of time. For example, the user may be exposed to a specific wavelength, for thirty minutes, immediately before sleeping.

In accordance with an embodiment, the user may, in the first week, expose themselves to blue light without shifting their sleep schedule. During the second week, the user may shift their sleep schedule for the week, either earlier or later (for example, one hour earlier or one hour later), and only sleep during the allotted times. During this second week, the user will continue to utilize the blue light therapy immediately before or after the shifted sleep. In subsequent weeks, the user may continue to utilize blue light therapy and gradually shift the sleep schedule, until a desired time is reached. Once the desired time is reached, the user may continue to use the blue light therapy in conjunction with the new sleep time for a predetermined amount of time (for example, three days or 1 week). In another embodiment, the user may attempt to sleep without the blue light, and the sleep records may be analyzed to provide a flag as to whether further blue light treatment is needed. At a point, the user may then cease to use the blue light therapy.

At one or more predetermined intervals, or based on one or more triggers, blue light therapy may be re-introduced to address circadian rhythm shifting. For example, the system may monitor for one or more indicators of circadian rhythm shifting. In an embodiment, elevated heart rate (for example, above 110 beats per minute) may be used as an indicator of circadian rhythm shifting. In another embodiment, determining that an individual is awake during predetermined night-time hours (such as, for example, 10 PM-6 AM) for more than a threshold amount of time (for example, 15 minutes, 30 minutes, 1 hour, or 1.5 hours) may be an indicator circadian rhythm shifting. The threshold may be for the aggregate period an individual is awake at night or for the longest single consecutive period an individual is awake.

In an embodiment, the system may include analyzing whether a user is in a sleep state or awake state. For example, the system may monitor for the presence of slow-waves via, for example, decreased heart rate, decreased blood pressure, reduced body temperature or any other suitable parameter. In another embodiment, a sleep diary may be utilized, logging or automatically logging information on a user's sleep patterns. In another embodiment, use of a user's device is monitored (e.g., email, games, etc.) to determine whether the user was awake and using the device.

For example, the system may analyze one or more of the following measurements, via a plurality of sensors determining sleep states and non-sleep states:

Time user goes to bed
Time user turns out lights intending to go to sleep
Time it takes user to fall asleep
Total amount of time awake during sleeping hours
Time user wakes up in the morning
How long user estimates they slept that night in total
Estimated time spent napping in the daytime

Additionally, sleep evaluation may be performed by collecting the following user data. Specifically, the user may be queried to answer the following queries:

| QUERY 1 | QUERY 2 | QUERY 3 | QUERY 4 |
|---|---|---|---|
| Determine whether | Determine whether the | Determine whether | Determine if the user |
| user has trouble falling | user has proper | user has trouble | is a night-shift |
| asleep at night | sleeping setup in their | staying awake in | worker |
| | room | the evening | |
| How many hours are | Is their bedroom | What time do they | Determine working |
| they awake during the | relatively cool at night | fall asleep? | hours at night, and |
| night? | (~60-67° F.)? | | hours available for |
| | | | sleep during the day |
| What time do they end | Do they keep lighting | What time do they | What time does the |
| up falling asleep? | dim in the evenings | wake up in the | user fall asleep in |
| | around bedtime (no | morning? | the morning? |
| | intense fluorescent | | |
| | lighting)? | | |
| How many hours do | Do they have | If they wake up | How long does it |
| they sleep? | distracting devices in | very early, do they | take for them to fall |
| | the room (e.g. ticking | stay awake the | asleep? |
| | clocks, flashing lights, | entire duration, or | |
| | sounds, smells, | do they fall back | |
| | moving objects, etc.)? | asleep? | |
| At what clock-time do | Is the room noisy at | Do they usually | Do they usually |
| they need to wake up? | night? | have restful sleep? | have restful sleep? |
| Do they usually have | Do they have a | What time do they | Do they have |
| restful sleep? | sleeping partner? If so, | need to wake up? | intermittent sleep? |
| | does the partner | | |
| | disturb their sleep? | | |
| How many alcoholic | | How many alcoholic | Do they experience |
| drinks/caffeinated | | drinks/caffeinated | sleep fatigue during |
| drinks/stimulants do | | drinks/stimulants do | work? |
| they take per day? | | they take per day? | |
| How many after 3 p.m.? | | How many after 3 p.m.? | |
| | | | Is their night-shift |
| | | | work expected to be |
| | | | chronic/long-term? |

In accordance with an embodiment, using answers provided from the above queries, the system may determine a preferred schedule for blue light therapy administration.

In an embodiment, the process may include: (1) logging sleep habits (including, for example, via automatic monitoring); (2) processing logged sleep data and providing output of sleep pattern changes over times; and (3) receiving a reminder to complete logging of sleep habits.

In accordance with an embodiment, depending on a determined sleep phase disorder, the user may expose themselves to blue light therapy during a specific clock-time and for a specific period of time, until their sleep period is shifted appropriately.

The process may include the following steps: (1) acclimating to the blue light in the daytime for a brief period of time (for example, about 5 minutes) before starting treatment; (2) collecting baseline sleep for one week, or any other suitable period of time, before blue light therapy is commenced; (3) developing a personalized therapy treatment plan; (4) administering, for a predetermined period of time (for example, thirty minutes), blue light in the morning upon waking up or the evening before sleeping, depending on whether the user's sleep cycle is shifted forward or backward.

The schedule may be implemented as follows: (1) the first 1-3 days of blue light therapy may occur during the same sleep schedule as from baseline, administering 30 minutes of blue light before or after sleep; (2) days 4-6 of blue light therapy will occur in a “shifted-sleep” schedule, where the user will be required to go to sleep a predetermined amount of time earlier or later (e.g., 1 hour) than their previous baseline sleep schedule, while still administering 30 min of blue light before or after sleep; (3) days 7-9 of blue light therapy will occur in a “shifted-sleep” schedule, where the user will further sleep, for example, 1 hour earlier or later than days 4-6, while still administering 30 min of blue light before or after sleep; and (4) days 10-17 will include the user keeping the last shifted-sleep schedule (either advanced or delayed), but without blue light therapy.

In one embodiment, peak transmission of the blue light therapy may occur at approximately 470 nm (460-480 nm), with an intensity at the cornea 2306 melanopiclux, 300 photopic lux, 3.0 W/m² at a distance of 40 cm and angle of exposure of 45°.

In a further embodiment, an attention-based engagement game or process may be used during administration of the blue light therapy, in order to maintain interest and focus on the therapy.

In accordance with the invention, a sleep tracker may be used to determine the efficacy of a blue light treatment. The sleep tracker may track sleep habits and quality, and collect user-based sleep information. Sleep data is then analyzed to determine if there is a significant circadian re-shift (such as, for example, one (1) hour or more), and the user may be prompted to perform a sleep hygiene/stimulant checklist, and potentially blue light treatment.

In one embodiment, an illustrative process includes: (1) receiving daily early morning awakenings (EMAs) for the sleep tracker; (2) receiving a text message/SMS reminder at a predetermined time, such as 45 minutes, before a scheduled sleep time; (3) providing one or more sleep-related tracking queries in the morning, upon wakeup; and (4) receiving weekly text message reminders on sleep hygiene tips.

In an embodiment of the present invention, the user may be provided with a video game they can play on their client device 102-106 for the software to determine whether the user is falling asleep.

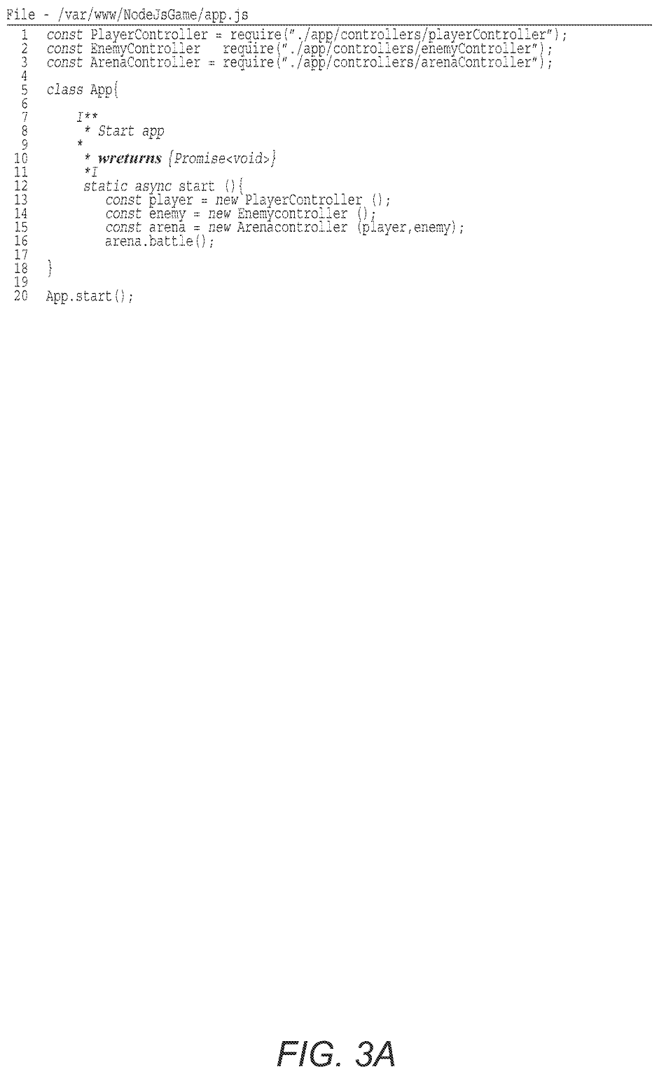

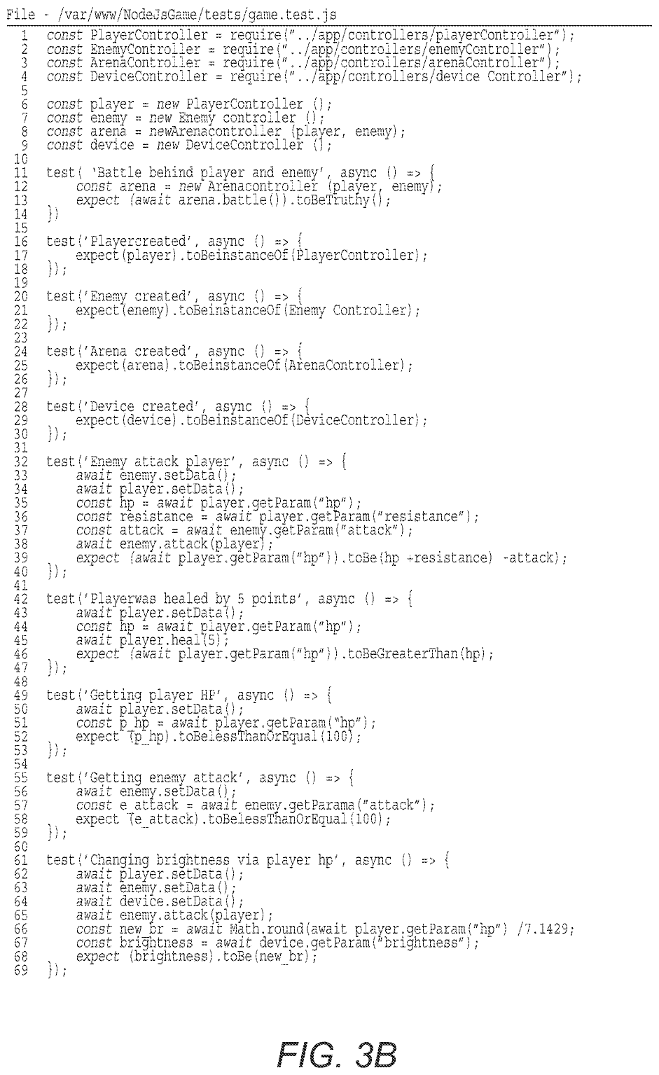

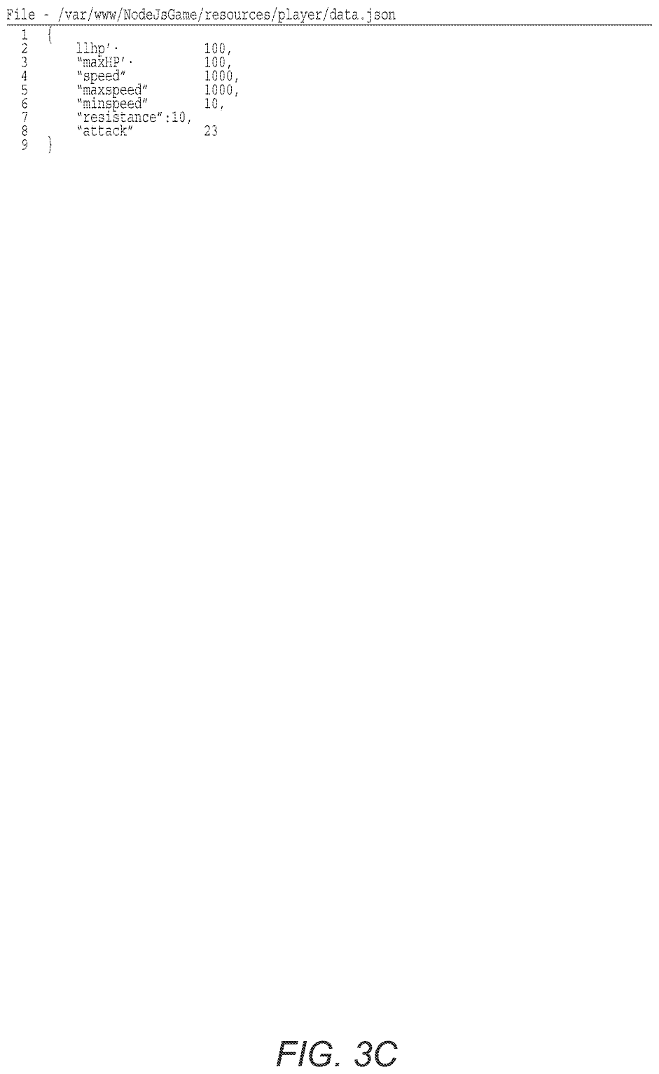

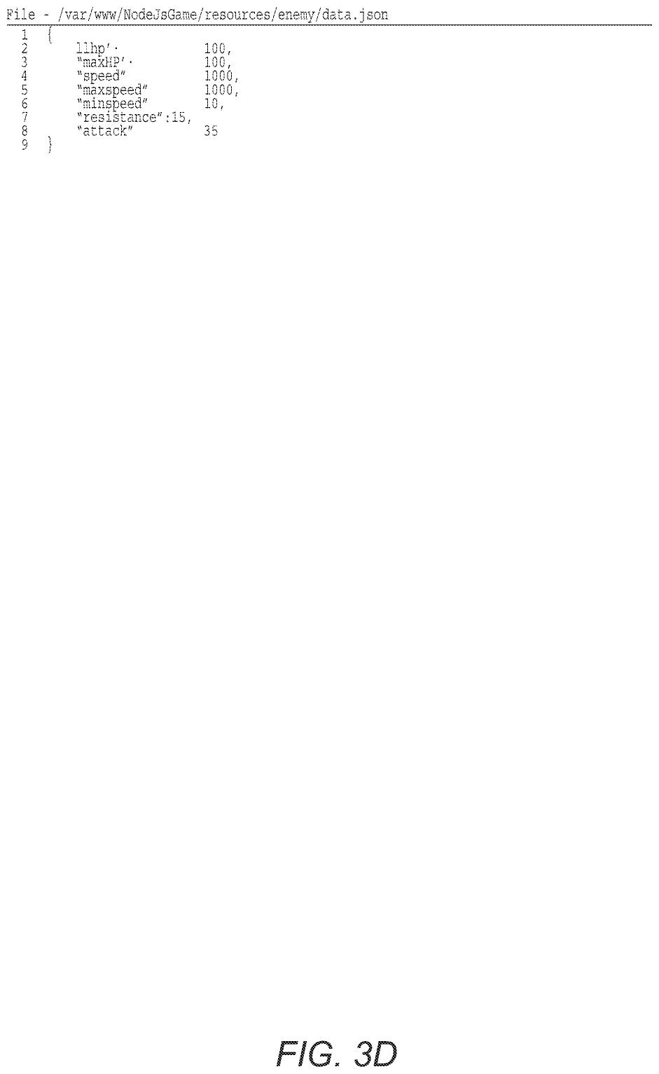

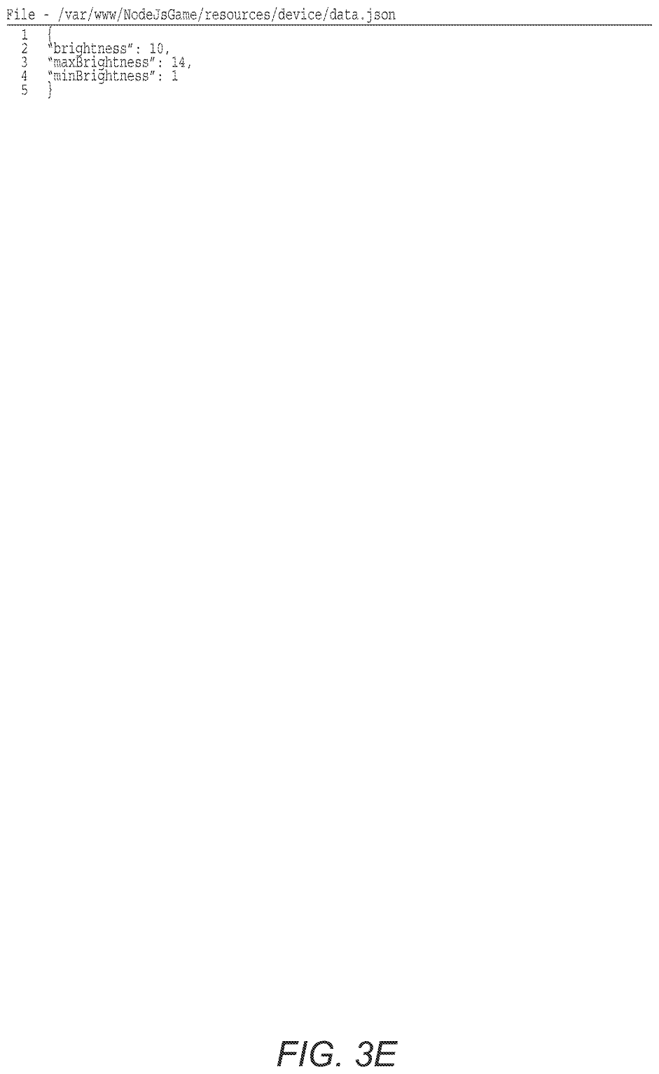

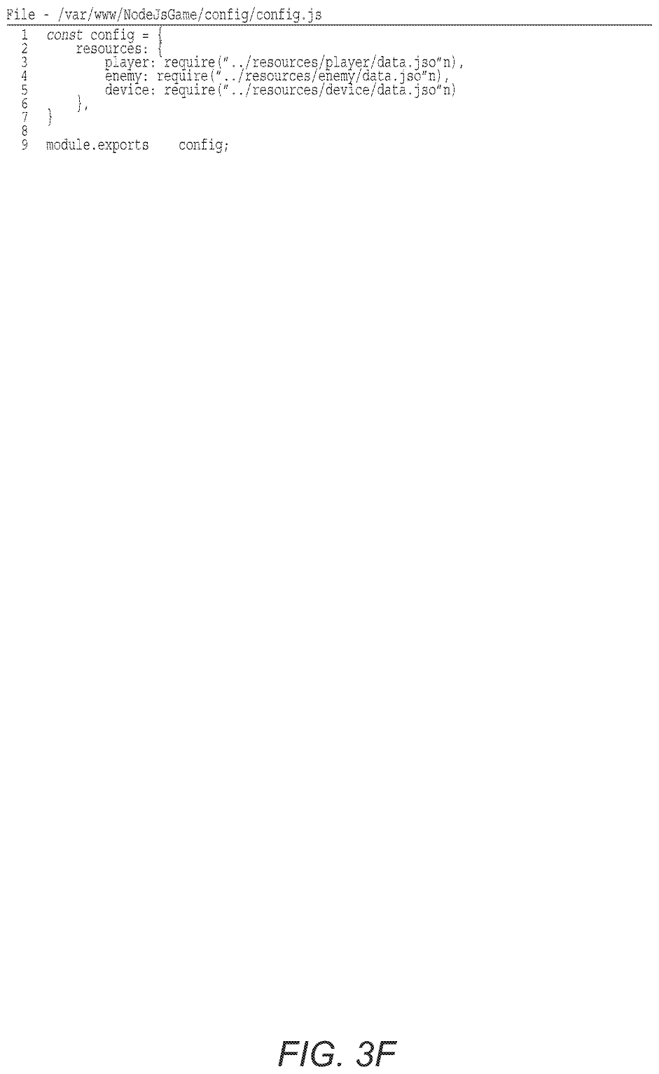

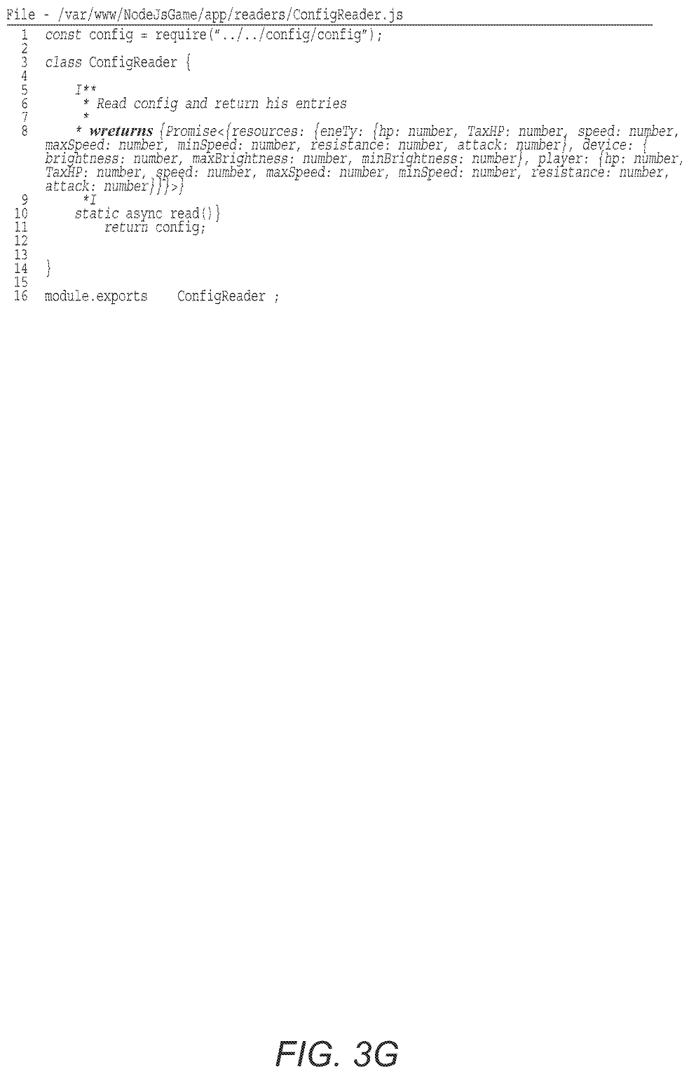

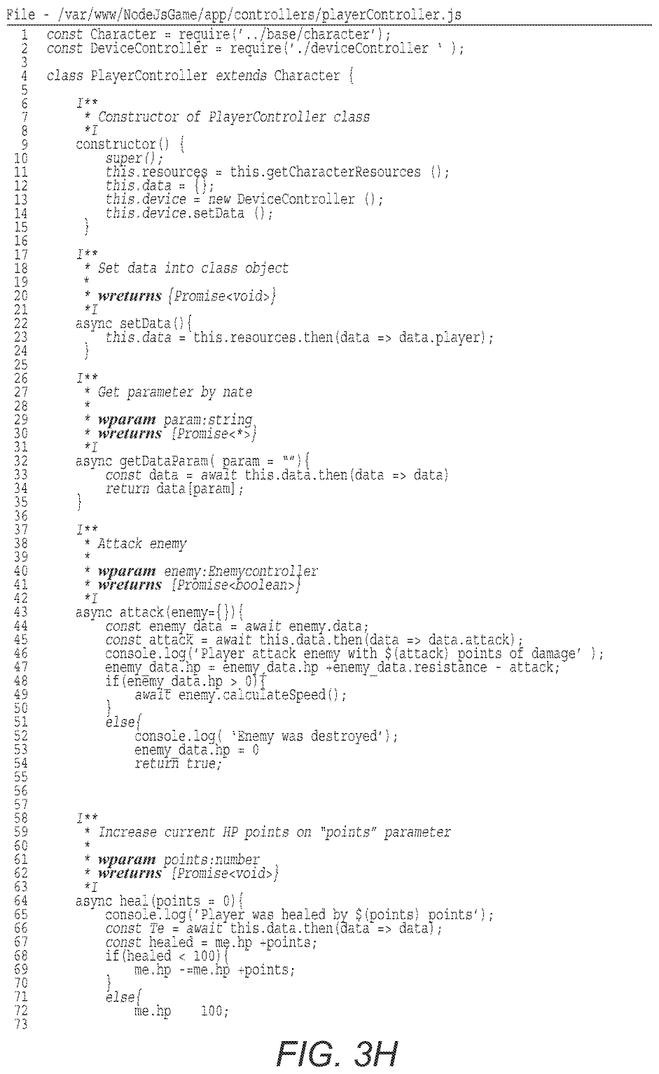

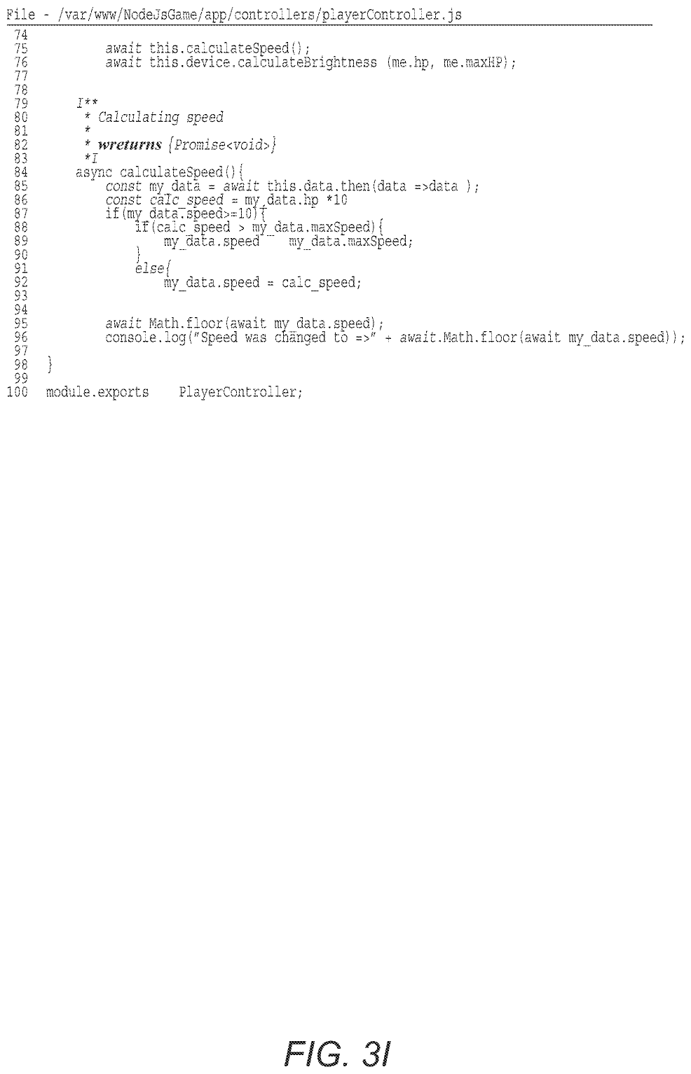

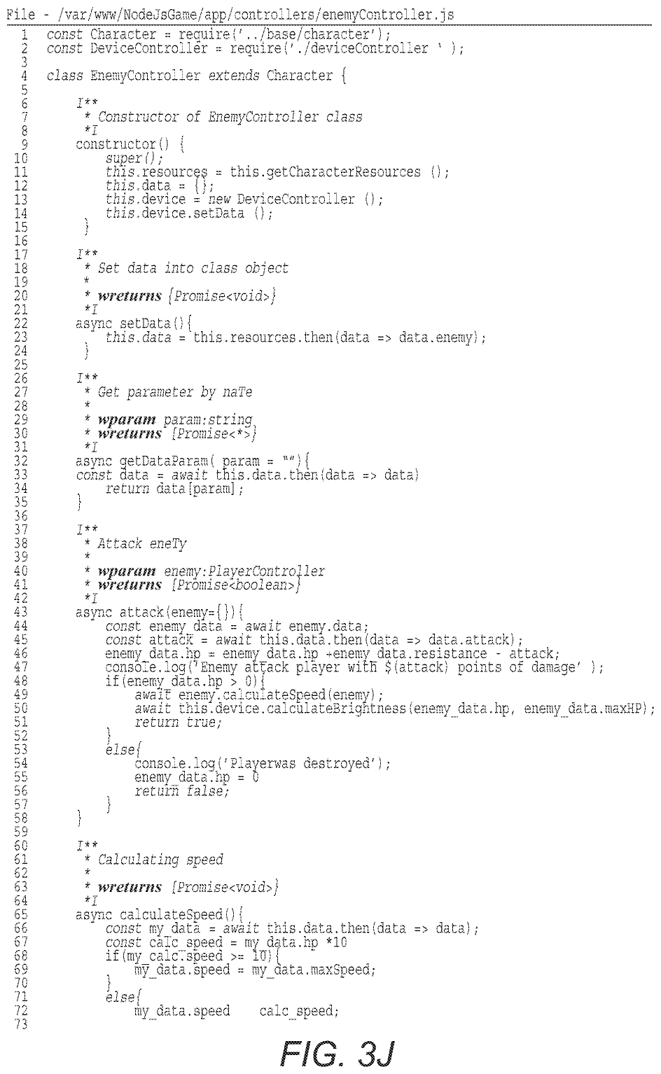

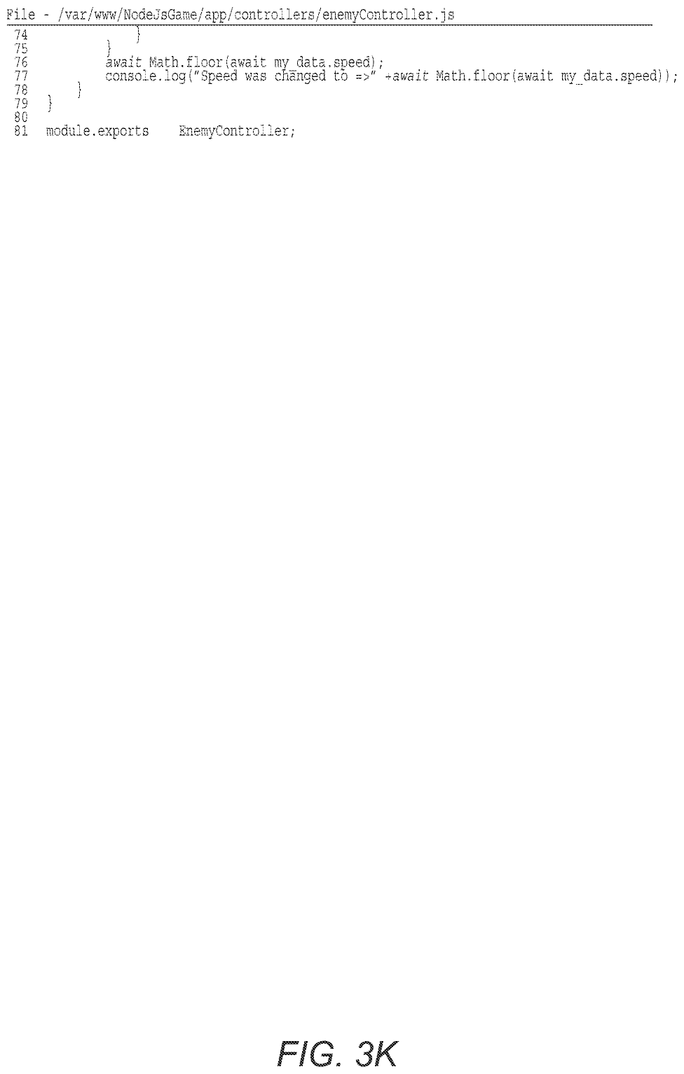

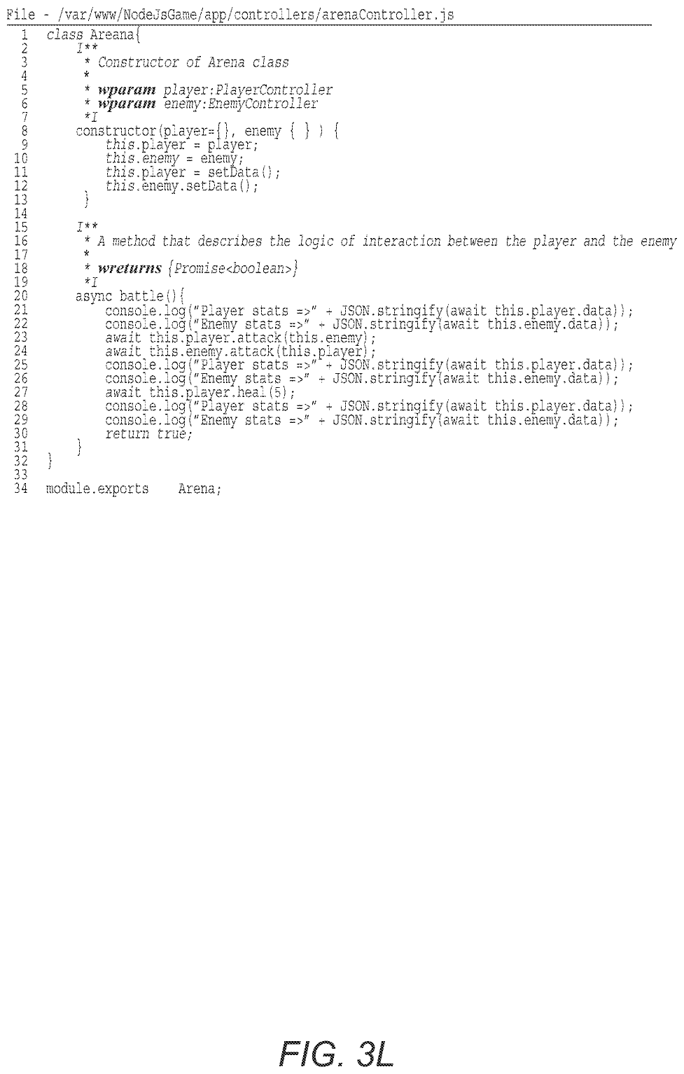

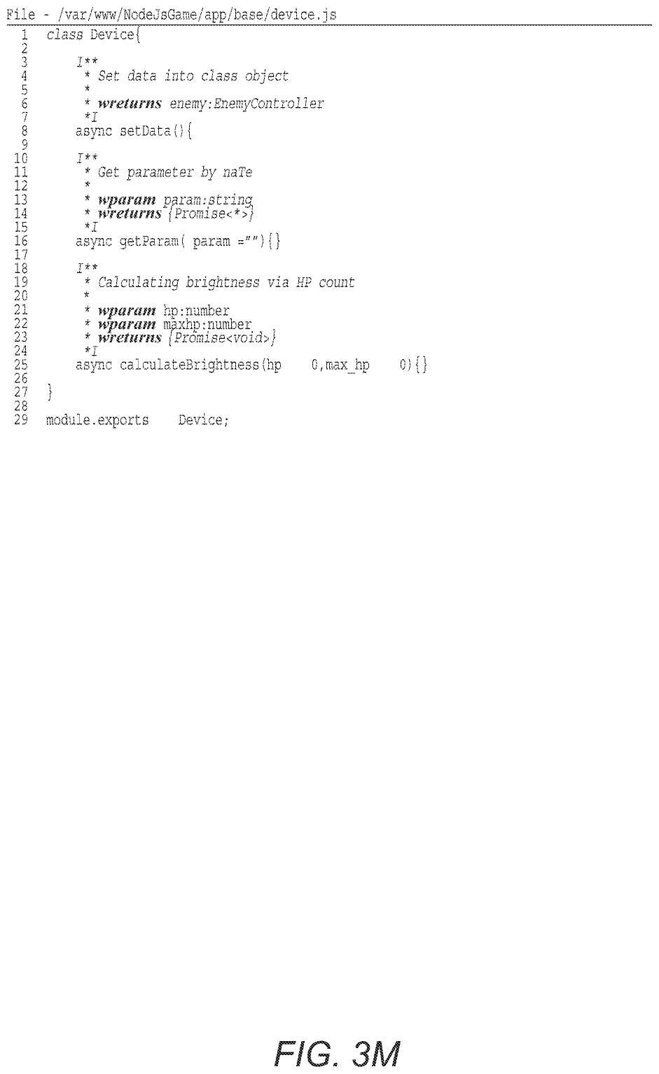

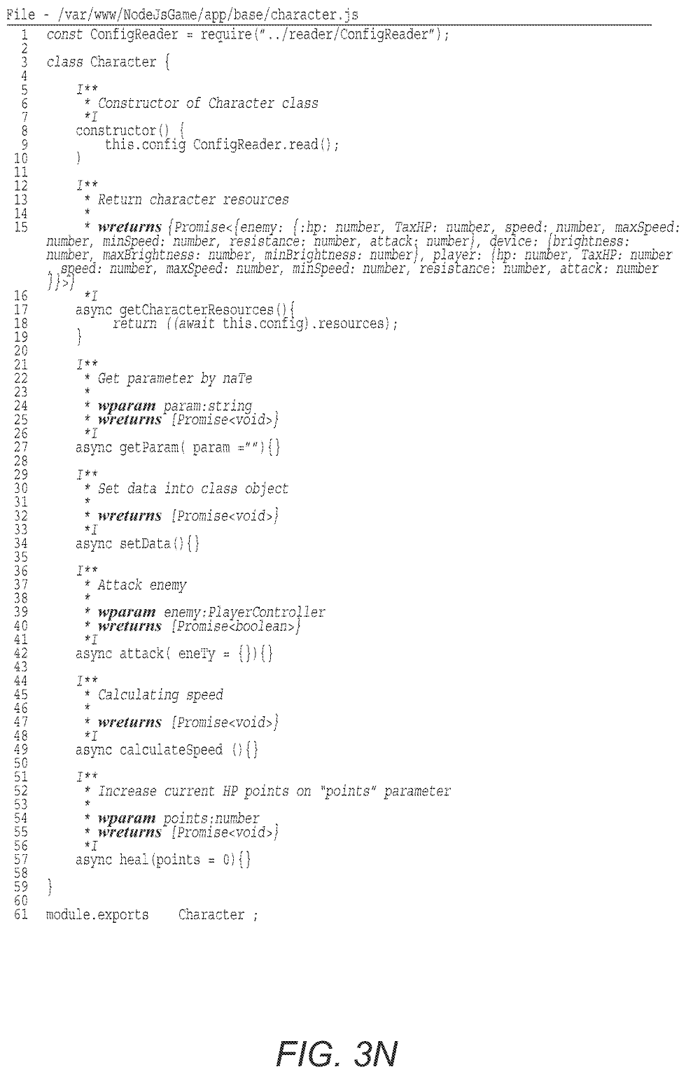

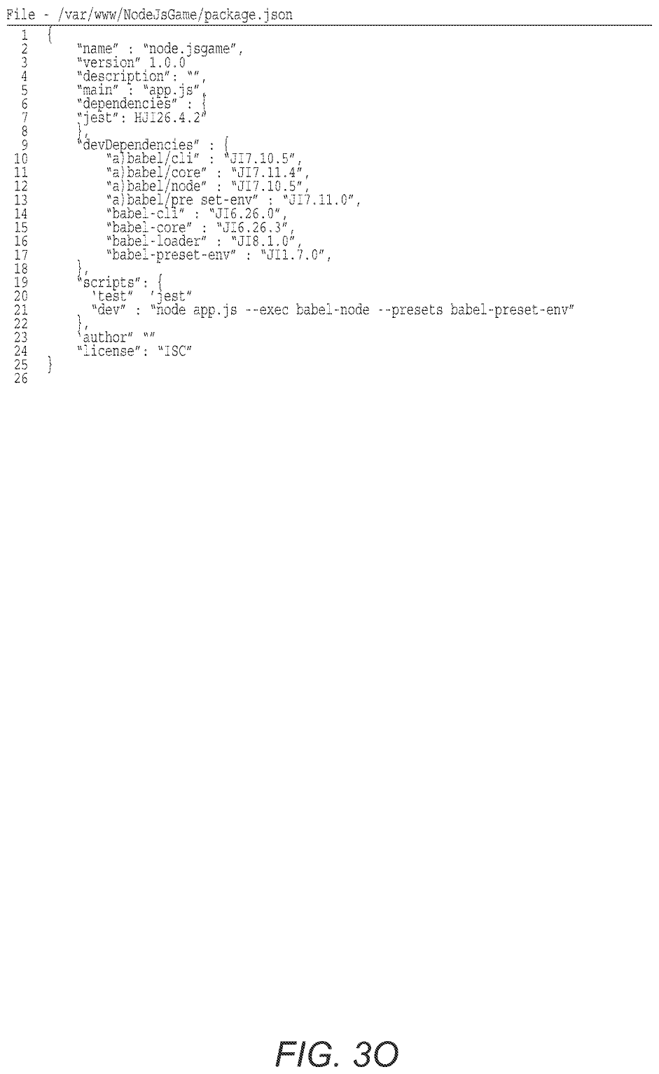

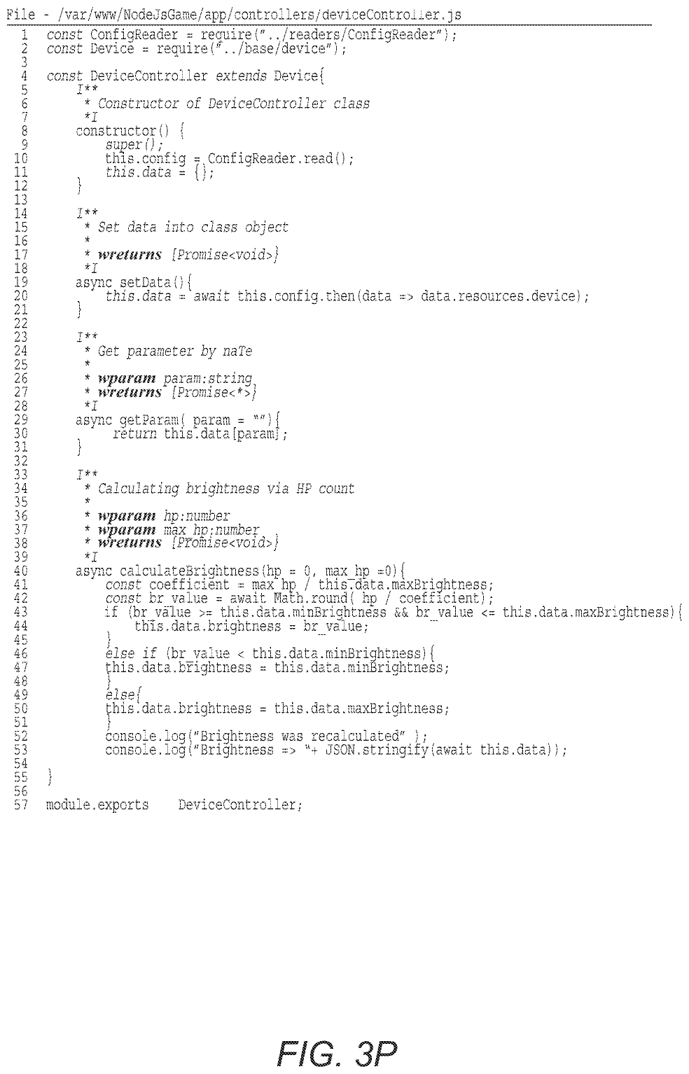

FIGS. 3A-3P show source code that can implement one or more aspects of an embodiment of the present invention. The figures include: (1) a first algorithm that increases game speed if player performance improves. Player performance is a value and can be set as an input value for the function in this algorithm. There are different parameters that affect a player's performance; and (2) a second algorithm that increases screen brightness if player performance worsens.

In certain embodiments, the therapy may be used as part of a treatment regimen, in conjunction with the use of one or more pharmaceutical compositions. Illustrative pharmaceutical compositions known for treating insomnia that may be used in conjunction with the therapy include: (a) GABA-A receptor Agonists, including benzodiazepines and benzodiazepine receptor agonists that act on GABA receptor sites and exert sedative, anxiolytic, muscle relaxant, and/or hypnotic effects, such as Zolpidem, Zaleplon and Eszopiclone; (b) Melatonin Receptor Agonists, such as Melatonin, Ramelteon and Tasimelteon; (c) Orexin Receptor Agonists, such as Suvorexant (in doses of, for example, 5 mg, 10 mg, 15 mg, or 20 mg); (d) Histamine-1 Receptor Antagonists, such as Doxepin (in doses of, for example, 3 mg and 6 mg; (e) Selective Serotonin Reuptake Inhibitors, such as mirtazapine, fluoxetine, citalopram and sertraline; (f) Tricylic antidepressants, such as doxepin, amitriptyline, and trimipramine; and (g) other suitable anti-depressants, anti convulsants or atypical antipsychotics.

While this invention has been described in conjunction with the embodiments outlined above, many alternatives, modifications and variations will be apparent to those skilled in the art upon reading the foregoing disclosure. Accordingly, the exemplary embodiments of the invention, as set forth above, are intended to be illustrative, not limiting. Various changes may be made without departing from the spirit and scope of the invention.

What is claimed is:

1. A computer system for provision of blue-light therapy comprising one or more processors, one or more computer-readable memories, and one or more computer-readable storage devices, and program instructions stored on at least one of the one or more storage devices for execution by at least one of the one or more processors via at least one of the one or more memories, the stored program instructions comprising:
determining a need for blue-light therapy; and
providing a blue-light stimulus to a user, wherein the blue-light stimulus comprises a visible range of 380 to 500 nm, and wherein, in response to the blue-light stimulus, a circadian rhythm of the user is shifted to a second rhythm, the second rhythm different than the circadian rhythm prior to the administration of the blue-light stimulus.

2. The blue-light therapy system according to claim 1, wherein the determining the need for blue-light therapy comprises determining a number of hours slept.

3. The blue-light therapy system according to claim 1, wherein the determining the need for blue-light therapy comprises monitoring at least one of a heart rate, pulse, blood pressure, body temperature, maximum rate of decline of body temperature, body movement patterns, mobility, or respiration rate, of the user.

4. The blue-light therapy system according to claim 1, wherein the blue-light stimulus comprises a visible range of approximately 470 nm.

5. The blue-light therapy system according to claim 1, wherein a duration of the blue-light stimulus is predetermined.

6. The blue-light therapy system according to claim 1, wherein a duration of the blue-light stimulus is 15 to 30 minutes.

7. The blue-light therapy system according to claim 1, wherein the blue-light stimulus is administered at a determined time.

8. The blue-light therapy system according to claim 7, wherein the determined time is 10 PM.

9. The blue-light therapy system according to claim 7, wherein the determined time is based on a sleep state of the user.

10. The blue-light therapy system according to claim 9, wherein the determined time is immediately after the user has awoken.

11. The blue-light therapy system according to claim 9, wherein the determined time is immediately prior to a beginning of the user's sleep cycle.

12. The blue-light therapy system according to claim 1, wherein, during the providing the blue-light stimulus to the user, it is determined, using a camera and the one or more processors, whether pupil constriction occurs in an eye of the user.

13. The blue-light therapy system according to claim 12, wherein, if it is determined that the pupil constriction occurs, a determination is made whether the pupil constriction is at a predetermined level.

14. The blue-light therapy system according to claim 13, wherein, if the pupil constriction is not at the predetermined level, a duration of the blue-light stimulus is lengthened.

15. The blue-light therapy system according to claim 13, wherein, if the pupil constriction is at a predetermined level, a duration of the blue-light stimulus is shortened.

16. The blue-light therapy system according to claim 3, wherein the blue-light stimulus is administered at a determined time and the determined time is based on the at least one of a heart rate, pulse, blood pressure, body temperature, maximum rate of decline of body temperature, body movement patterns, mobility, or respiration rate, of the user.

17. The blue-light therapy system according to claim 1, wherein the blue-light stimulus comprises a visible range of 380 to 450 nm.

18. The blue-light therapy system according to claim 1, wherein the blue-light stimulus comprises a visible range of 450 to 500 nm.

19. A computer implemented method for blue-light therapy, the method comprising:
determining a need for blue-light therapy; and providing a blue-light stimulus to a user, wherein the blue-light stimulus comprises a visible range of 380 to 500 nm, and wherein, in response to the blue-light stimulus, a circadian rhythm of the user is shifted to a second rhythm, the second rhythm different than the circadian rhythm prior to the administration of the blue-light stimulus.
